(19) United States
(12) Patent Application Publication
(10) Pub. No.: US 2008/0097346 A1
(43) Pub. Date: Apr. 24, 2008

(54) Trocar cannula

(75) Inventor: Steven T. Charles, Memphis, TN (US)

Correspondence Address: ALCON, IP LEGAL, TB4-8, 6201 SOUTH FREEWAY, FORT WORTH, TX, 76134, US

(73) Assignee: Alcon, Inc.

(21) Appl. No.: 11/523,765

(22) Filed: Sep. 19, 2006

(51) Int. Cl. A61M 5/00

(52) U.S. Cl. 604/264

(57) ABSTRACT

A trocar cannula having a tube and a hub. The hub is attached to the proximal end of the tube so that the tube may be inserted into an incision. The proximal side of the hub is rounded, curved or slanted so as to minimize contact with the tissue surrounding the incision.

This invention relates to ophthalmic surgical equipment and more particularly to posterior segment ophthalmic surgical equipment.

### BACKGROUND OF THE INVENTION

Microsurgical instruments typically are used by surgeons for removal of tissue from delicate and restricted spaces in the human body, particularly in surgery on the eye, and more particularly in procedures for removal of the vitreous body, blood, scar tissue, or the crystalline lens. Such instruments include a control console and a surgical handpiece with which the surgeon dissects and removes the tissue. The handpiece has a surgical tool such as a vitreous cutter probe or an ultrasonic fragmenter for cutting or fragmenting the tissue and is connected to the control console by a long air pressure (pneumatic) line or power cable and by long conduits, cable, optical cable or flexible tubes for supplying an infusion fluid to the surgical site and for withdrawing or aspirating fluid and cut/fragmented tissue from the site. The cutting, infusion and aspiration functions of the handpiece are controlled by the remote control console that not only provides power for the surgical handpiece(s) (e.g., a reciprocating or rotating cutting blade or an ultrasonically vibrated needle), but also controls the flow of infusion fluid and provides a source of reduced pressure (relative to atmosphere) for the aspiration of fluid and cut/fragmented tissue. The functions of the console are controlled manually by the surgeon, usually by means of a foot-operated switch or proportional control.

During posterior segment surgery, the surgeon typically uses several instruments during the procedure. This requires that these instruments be inserted into, and removed out of the incision. This repeated removal and insertion can cause trauma to the eye at the incision site. To address this concern, trocar cannulae were developed at least by the mid-1980s. These devices consist of a narrow tube with an attached hub. The tube is inserted into the incision up to the hub, which acts as a stop, preventing the tube from entering the eye completely. Surgical instruments can be inserted into the eye through the tube, and the tube protects the incision from repeated contact by the instruments. In addition, the surgeon can use the instrument, by manipulating the instrument when the instrument is inserted into the eye through the tube, to help position the eye during surgery. Prior art trocar cannulae have cylindrical hubs with a large contact area of the hub with the sclera at the wound site. This large contact area increases the amount of effort required to move the instruments in an angular fashion with respect to the scleral surface because the normally rounded scleral surface must be indented and then applanted after the cannula bottom is rotated enough to make contact with the sclera, with a corresponding increase in bending moment on the instrument shaft, increasing the risk of instrument flexion. In addition, existing cannulae often get pulled out of the incision when rotated at a large angle to the surface of the eye.

Accordingly, a need continues to exist for a trocar cannula that provides easier manipulation and rotation and that resists being pulled from the incision.

### BRIEF SUMMARY OF THE INVENTION

The present invention improves upon prior art by providing a trocar cannula having a tube and a hub. The hub is attached to the proximal end of the tube so that the tube may be inserted into an incision. The proximal side of the hub is rounded, curved or slanted so as to minimize contact with the tissue surrounding the incision.

Accordingly, an objective of the present invention to provide a trocar cannula.

Another objective of the present invention to provide a trocar cannula that minimizes tissue contact.

A further objective of the present invention to provide a trocar cannula with a rounded, curved or slanted hub.

Other objectives, features and advantages of the present invention will become apparent with reference to the drawings, and the following description of the drawings and claims.

### BRIEF DESCRIPTION OF THE DRAWINGS

FIG. 1 is an enlarged cross-section view of a prior art trocar cannula.

FIG. 2 is an enlarged cross-section view of a first embodiment of the trocar cannula of the present invention.

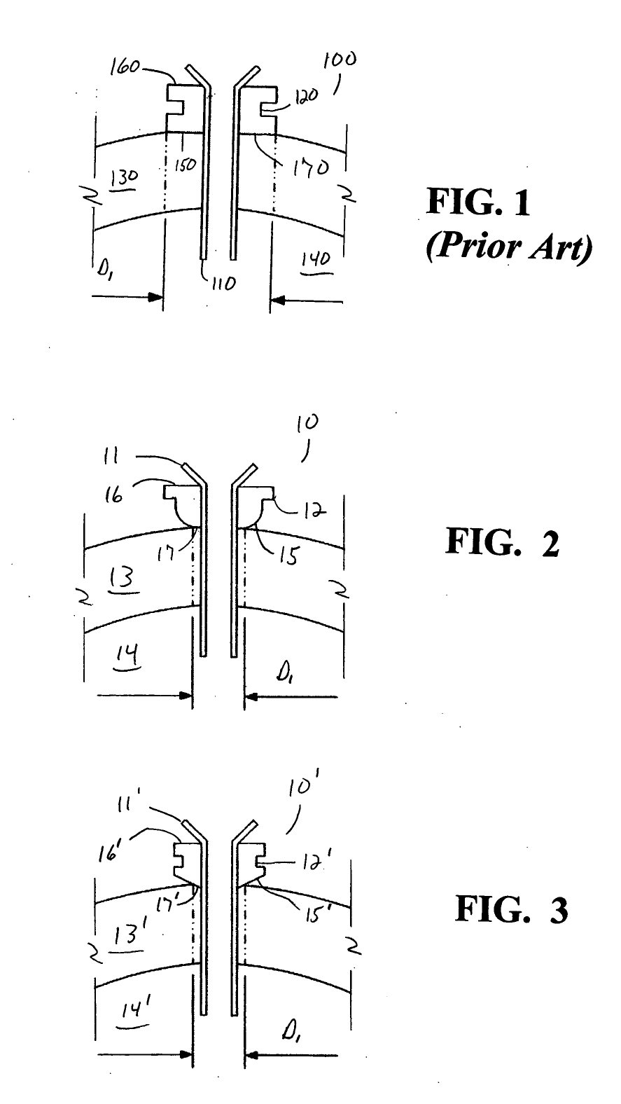

FIG. 1 is an enlarged cross-section view of a second embodiment of the trocar cannula of the present invention.

### DETAILED DESCRIPTION OF THE INVENTION

As best seen in FIG. 1, prior art trocar cannula 100 consists of tube 110 and hub 120. Tube 110 is of sufficient length to extend through sclera 130 and enter posterior chamber 140. Hub 120 is generally cylindrical, with flat distal face 150 and proximal face 160. Flat distal face 150 causes the diameter D₁ of contact area 170 to be relatively large, on the order of 2.3 mm or larger.

As seen in FIG. 2, in a first embodiment of the present invention, trocar cannula 10 consists of tube 11 and hub 12. Tube 11 is of sufficient length to extend through sclera 13 and enter posterior chamber 14. Tube 11 and hub 12 are made from any suitable material such as stainless steel, titanium or thermoplastic. Hub 12 is generally cylindrical, with flat proximal face 16 and curved or rounded distal face 15. Rounded distal face 15 causes the diameter D₁ of contact area 17 to be relatively small, on the order of 0.9 mm.

Alternatively, as seen in FIG. 3, trocar cannula 10 consists of tube 11′ and hub 12′. Tube 11′ is of sufficient length to extend through sclera 13′ and enter posterior chamber 14′. Tube 11′ and hub 12′ are made from any suitable material such as stainless steel, titanium or thermoplastic. Hub 12′ is generally cylindrical, with flat proximal face 16′ and angled or sloped distal face 15′. Angled or sloped distal face 15′ also causes the diameter D₁ of contact area 17′ to be relatively small, on the order of 0.9 mm.

While certain embodiments of the present invention have been described above, these descriptions are given for purposes of illustration and explanation. Variations, changes, modifications and departures from the systems and methods disclosed above may be adopted without departure from the scope or spirit of the present invention.

I claim:

1. A trocar cannula, comprising:
a) a tube; and
b) a hub connected to the tube, the hub having a proximal face and a distal face, the distal face being curved or rounded.

2. A trocar cannula, comprising:
a) a tube; and
b) a hub connected to the tube, the hub having a proximal face and a distal face, the distal face being angled or slanted.
